(19) United States
(12) Patent Application Publication
(10) Pub. No.: US 2021/0307376 A1
(43) Pub. Date: Oct. 7, 2021

(54) Compositions Comprising Nicotine and/or Nicotine Salts and Ultrasonic Aerosolisation of Compositions Comprising Nicotine and/or Nicotine Salts

(71) Applicant: SHAHEEN INNOVATIONS HOLDING LIMITED, Abu Dhabi (AE)

(72) Inventors: Imad Lahoud, Abu Dhabi (AE); Mohammed Alshaiba Saleh Ghannam Almazrouei, Abu Dhabi (AE); Jeffrey Machovec, Abu Dhabi (AE)

(21) Appl. No.: 17/220,189

(22) Filed: Apr. 1, 2021

(30) Foreign Application Priority Data
Apr. 6, 2020 (EP) EP20168231.7

(51) Int. Cl. A24B 15/167; A24F 40/05; B05B 17/06; A24F 40/44; A24F 40/10

(52) U.S. Cl. CPC A24B 15/167; A24F 40/05; A24F 40/10; A24F 40/44; B05B 17/0684

(57) ABSTRACT

The present invention relates to compositions comprising nicotine and/or salts of nicotine, in particular compositions comprising nicotine and/or salts of nicotine for ultrasonic aerosolisation.

### CROSS-REFERENCE TO RELATED APPLICATION

The present application claims the benefit of priority to European Patent Application No. 20168231.7, filed on 6 Apr. 2020, the entire contents of which are incorporated by reference herein.

### FIELD OF THE INVENTION

The present invention relates to compositions comprising nicotine and/or nicotine salts. The present invention also relates to ultrasonic aerosolisation of compositions comprising nicotine and/or nicotine salts.

### BACKGROUND OF THE INVENTION

Electronic nicotine delivery systems (“ENDS”) provide an alternative to smoking combustible cigarettes. Their rise in popularity is due, in part, to their ability to deliver nicotine and its associated satisfaction to their users.

Some users prefer relatively high levels of nicotine in the composition inhaled from their devices, to achieve their desired level of satisfaction. Preferably, the high level of nicotine in the composition is from 40 to 60 mg/ml, optionally 50 mg/ml. High levels of nicotine in an inhaled vapour, produced by ENDS, can produce a sensory irritation commonly known as “throat hit” that users find unpleasant. In recent years, the development of “nicotine salts” has permitted providers to raise the level of nicotine in ENDS to more than twice the highest concentrations found on the market in the early years of ENDS.

The rise in popularity of “nicotine salts” in ENDS can be attributed to their performance of two essential functions: one, these nicotine salts reduce the throat hit sensation felt by the user; and, two, enhance the pharmacologic effect of the nicotine by enhancing nicotine uptake into the bloodstream.

Previously, all ENDS found on the market relied on a heated coil system to vaporize their nicotine containing liquid. Recently, a new class of ENDS that produces an inhalable aerosol via ultrasonic vibrations has been developed and continues to evolve. One such device utilising ultrasonic vibrations has been developed by Shaheen Innovations Holding Limited and is described in PCT application number PCT/IB2019/060810 (the disclosure of which is hereby incorporated by reference in its entirety).

One advantage of these new devices utilising ultrasonic vibrations is that they are able to produce a vapour-like aerosol without heating the nicotine containing liquid. It has been found that these devices utilising ultrasonic vibrations are able to deliver nicotine at an even higher rate than heated coil ENDS because the absence of heat prevents denaturisation of the nicotine molecules and salts during aerosolisation.

### SUMMARY OF THE INVENTION

The present invention relates in some non-limiting aspects to aerosolisation of compositions comprising nicotine and/or salts of nicotine, the aerosolisation utilising ultrasonic vibrations.

The present invention is as set out in the claims. In particular, representative features of the present invention are set out in the following clauses, which stand alone or may be combined, in any combination, with one or more features disclosed in the text of the specification:

1. An e-liquid composition for use with an ultrasonic device, comprising:
a nicotine salt.

2. The e-liquid composition of clause 1, wherein the e-liquid composition further comprises one, two, three or four of:
propylene glycol;
vegetable glycerin:
water; and,
flavourings.

3. The e-liquid composition of clause 2, wherein the relative amount of vegetable glycerin in the composition is: from 55 to 80% (w/w), or from 60 to 80% (w/w), or from 65 to 75% (w/w), or 70% (w/w).

4. The e-liquid composition of clause 2 or clause 3, wherein the relative amount of propylene glycol in the composition is: from 5 to 30% (w/w), or from 10 to 30% (w/w), or from 15 to 25% (w/w), or 20% (w/w).

5. The e-liquid composition of any one of clauses 2 to 4, wherein the relative amount of water in the composition is: from 5 to 15% (w/w), or from 7 to 12% (w/w), or 10% (w/w).

6. The e-liquid composition of any one of clauses 2 to 5, wherein the amount of nicotine and/or nicotine salt in the composition is: from 0.1 to 80 mg/ml, or from 0.1 to 50 mg/ml, or from 1 to 25 mg/ml, or from 10 to 20 mg/ml, or 17 mg/ml.

7. The e-liquid composition of any one of clauses 2 to 6, wherein the composition comprises, or consists of, (in % (w/w)):

| | |
|---|---|
| propylene glycol | from 10 to 20 |
| vegetable glycerin | from 65 to 75 |
| water | from 5 to 15 |
| nicotine | from 1 to 5 |
| organic acid | from 0.1 to 5.0 |
| flavourings | balance; |

wherein the organic acid comprises, or consists of: benzoic acid, levulinic acid, malic acid, tartaric acid, salicylic acid, citric acid or lactic acid, or any combination of any two, three, four, five, six of seven of these organic acids.

8. The e-liquid composition of any one of clauses 2 to 7, wherein the composition comprises (in % (w/w)):

| | |
|---|---|
| organic acid | from 0.1 to 5.0; or, from 1.0 to 4.0; or, from 0.1 to 2.0. |

9. The e-liquid composition of any one of clauses 2 to 8, wherein the composition comprises, or consists of, (in % (w/w)):

| | |
|---|---|
| propylene glycol | from 11 to 16 |
| vegetable glycerin | from 69 to 71 |
| water | from 9 to 11 |
| nicotine | from 1 to 3 |
| levulinic acid | from 0.1 to 4.0 |
| flavourings | balance. |

10. The e-liquid composition of clause 9, wherein the composition comprises (in % (w/w)):

| | |
|---|---|
| propylene glycol | from 14 to 16. |

11. The e-liquid composition of any one of clauses 1 to 10, wherein the composition comprises (in % (w/w)):

| | |
|---|---|
| levulinic acid | from 1.0 to 4.0; or, from 0.1 to 1.0; or, from 0.1 to 0.5. |

12. The e-liquid composition of any one of clauses 2 to 11, wherein the composition comprises (in % (w/w)):

| | |
|---|---|
| propylene glycol | 15.1 |
| vegetable glycerin | 70 |
| water | 10 |
| nicotine | 1.7 |
| levulinic acid | 0.2 |
| flavourings | 3; or, |
| propylene glycol | 12.87 |
| vegetable glycerin | 70 |
| water | 10 |
| nicotine | 1.7 |
| levulinic acid | 2.43 |
| flavourings | 3; or, |
| propylene glycol | 14.08 |
| vegetable glycerin | 70 |
| water | 10 |
| nicotine | 1.7 |
| levulinic acid | 1.22 |
| flavourings | 3; or, |
| propylene glycol | 11.64 |
| vegetable glycerin | 70 |
| water | 10 |
| nicotine | 1.7 |
| levulinic acid | 3.66 |
| flavourings | 3. |

13. The e-liquid composition of any one of clauses 1 to 12, wherein the nicotine salt is selected from the group consisting of:
nicotine benzoate, nicotine lactate, nicotine maleate, nicotine ditartrate, nicotine salicylate, nicotine citrate and nicotine levulinate, or any combination of any two, three, four, five, six or seven of these nicotine salts.

14. The e-liquid composition of any one of clauses 1 to 13, wherein the nicotine salt is nicotine levulinate.

15. The e-liquid composition of clause 13 or clause 14, wherein the molar ratio of nicotine to organic acid salt (nicotine:organic acid salt) is: 1:1 or greater; or, 1:2 or greater; or, from 1:1 to 1:4; or, from 1:1 to 1:3.

16. The use of an e-liquid composition of any one of clauses 1 to 15 in providing nicotine to a user, the use comprising:
providing an e-liquid composition according to any one of clauses 1 to 15;
placing the e-liquid composition in an ultrasonic device; and,
aerosolising the e-liquid composition in the ultrasonic device.

17. The use of clause 16, wherein the use further comprises:
is inhaling the aerosolised composition.

18. The use of clause 16 or clause 17, wherein the ultrasonic device is an ultrasonic mist inhaler, comprising:
a liquid reservoir structure comprising a liquid chamber adapted to receive liquid to be atomized,
a sonication chamber in fluid communication with the liquid chamber,
a capillary element arranged between the liquid chamber and the sonication chamber.

19. The use of clause 18, wherein the capillary element is a material at least partly in bamboo fibers.

20. The use of clause 19, wherein the capillary element material is 100% bamboo fiber; or, wherein the capillary element material is at least 75% bamboo fiber and, preferably, 25% cotton.

21. The use of clause 19 or clause 20, wherein the capillary element is of a thickness between 0.27 mm and 0.32 mm and, preferably, has a density between 38 g/m² and 48 g/m².

22. The use of any one of clauses 19 to 21, wherein the capillary element has a flat shape.

23. The use of any one of clauses 19 to 22, wherein the capillary element comprises a central portion and a peripheral portion.

24. The use of any one of clauses 19 to 23, wherein the peripheral portion has an L-shape cross section extending down to the liquid chamber.

25. The use of any one of clauses 19 to 24, wherein the central portion has a U-shape cross section extending down to the sonication chamber.

26. A method of delivering a nicotine salt to a user, the method comprising:
providing an e-liquid composition according to any one of clauses 1 to 15;
placing the e-liquid composition in an ultrasonic device; and,
aerosolising the e-liquid composition in the ultrasonic device.

27. The method of clause 26, wherein the method further comprises:
inhaling the aerosolised composition.

### DETAILED DESCRIPTION OF THE INVENTION

Embodiments of the present disclosure will be described more fully hereinafter. Embodiments of the claims may, however, be embodied in many different forms and should not be construed as limited to the embodiments set forth herein. The examples set forth herein are non-limiting examples and are merely examples among other possible examples.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

“Aerosol” refers to a suspension of solid particles or liquid droplets in air or another gas. The aerosol produced by ENDS includes liquid droplets comprising nicotine, and other components, in air, which in use is inhaled by a user.

“Bioactive” refers to a compound that has an effect on a living organism.

“E-liquid” refers to a flavoured or non-flavoured fluid used in an electronic cigarette, ENDS or similar device.

“ENDS” refers to electronic nicotine delivery systems. ENDS provide an alternative to smoking combustible cigarettes. Common ENDS on the market utilise heated coil systems to vaporize their nicotine containing liquid. A new class of ENDS produces an inhalable aerosol via ultrasonic vibrations.

“Freebase nicotine” refers to an unprotonated nicotine molecule.

“Nicotine salt” refers to salts of nicotine including, but not limited to, nicotine benzoate, nicotine lactate, nicotine malate, nicotine ditartrate, nicotine salicylate, nicotine citrate and nicotine levulinate.

“Off-gassed” refers to when a volatile compound is released into the air.

“Weak acid” (for example a “weak organic acid) refers to an acid that only partially dissociates into its ions in an aqueous solution compared to a “strong acid” that fully dissociates into its ions.

“% (w/w)” refers to the amount of a component present “weight for weight”, i.e. the proportion of a particular substance within a composition or mixture, as measured by weight.

Nicotine Delivery

Due to the effectiveness of nicotine delivery in ultrasonic devices and the increased sensory irritation that occurs with nicotine levels above 6 mg/ml, nicotine salts are desirable for use in ultrasonic devices.

Nicotine salts are formed by combining freebase nicotine, a basic molecule, with a weak organic acid (for example, but not limited to, benzoic acid, levulinic acid, malic acid, tartaric acid, salicylic acid, citric acid and lactic acid).

Combining nicotine with a weak organic acid, for example in aqueous solution, lowers the pH and changes the freebase (or unprotonated) nicotine molecule to one of two protonated forms: monoprotonated and diprotonated (Reaction Scheme 1).

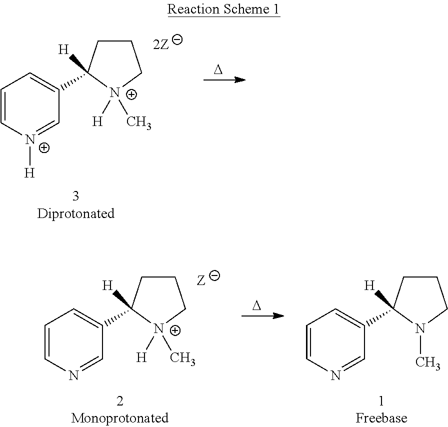

In Reaction Scheme 1, Z⁻ is the counter anion formed from deprotonation of the corresponding weak organic acid.

In the monoprotonated form, one of the two nitrogen atoms in the nicotine molecule acquires a proton from the acid and becomes ionised. In the diprotonated form, both nitrogen atoms of the nicotine molecule are protonated. It is thought that this pH reduction and subsequent protonation of the nicotine is what leads to the reduction in harshness when a nicotine salt is inhaled by a user.

Nicotine salts include nicotine benzoate, nicotine lactate, nicotine malate, nicotine ditartrate, nicotine salicylate, nicotine citrate and nicotine levulinate. All of these salts are created such that they exist in a monoprotonated form in an e-liquid. Their effectiveness in both throat hit reduction and nicotine uptake in the body have been studied and vary from salt to salt, in heated coil systems. The present inventors have found that similar variations in effectiveness occur in ultrasonic devices, but to a surprisingly different extent.

E-Liquids

Typical e-liquids comprise nicotine (optionally in the form of nicotine salts), flavourings, propylene glycol and a vegetable glycerin.

Typical e-liquids comprise from 57 to 69% (w/w) vegetable glycerin and from 30 to 42% (w/w) propylene glycol. The balance is formed of water, nicotine and/or nicotine salts, along with any flavourings. Optionally, the amount of nicotine (optionally in the form of nicotine salts) in e-liquids is from 0.1 to 80 mg/ml, or from 0.1 to 50 mg/ml.

In the present invention, the e-liquid comprises vegetable glycerin, propylene glycol and water. The balance is formed of nicotine and/or nicotine salts, along with any flavourings.

Optionally, the relative amount of vegetable glycerin in the e-liquid is: from 55 to 80% (w/w), or from 60 to 80% (w/w), or from 65 to 75% (w/w), or at least 70% (w/w).

Optionally, the relative amount of propylene glycol in the e-liquid is: from 5 to 30% (w/w), or from 10 to 30% (w/w), or from 15 to 25% (w/w), or at least 20% (w/w).

Optionally, the nicotine and/or nicotine salts, along with any flavourings are included as part of the total % (w/w) of the propylene glycol relative amount.

Optionally, the relative amount of water in the e-liquid is: from 5 to 15% (w/w), or from 7 to 12% (w/w), or at least 10% (w/w).

Optionally, the relative amount of nicotine in the e-liquid is: from 0.1 to 80 mg/ml, from 0.1 to 50 mg/ml, or from 1 to 25 mg/ml, or from 10 to 20 mg/ml, or at least 17 mg/ml.

In typical e-liquid compositions, if the vegetable glycerin % (w/w) is decreased, the % (w/w) of propylene glycol increases proportionally. In one non-limiting example, when the vegetable glycerin is present at 50% (w/w), propylene glycol is present at 40% (w/w) and water is present at 10% (w/w), there is a reduction in the amount of vapour the e-liquid produces. Vegetable glycerin is the predominant vapour “cloud” producer in the mixture, and it is preferable to maintain the vegetable glycerin at or above 50% (w/w).

Heated coil systems such as JUUL use a resistive coil wire to heat an e-liquid to approximately 215° C. (Talih et al.). At these temperatures (above 200° C.) the nicotine salt undergoes a process called disproportionation which yields, for two molecules of monoprotonated nicotine, one molecule of diprotonated nicotine and one molecule of unprotonated (freebase) nicotine (Seeman et al.).

For a compound to be considered bioactive, it is required to have an effect on a living organism. The protonated forms of nicotine cannot easily pass through the lipid bilayer of cell membranes, and therefore it is difficult for the protonated form of nicotine to transfer into the bloodstream and then travel to the brain, where it will have a biological effect on the person by binding to the nicotinic acetylcholine receptors in the brain. As a result, protonated forms of nicotine are not considered bioactive. The protonated forms of nicotine cannot easily pass through the alveoli in the lungs into the bloodstream because protonated forms of nicotine are not very soluble in lipids. The generation of diprotonated nicotine further reduces the amount of nicotine that can be quickly delivered into the bloodstream. To the contrary, freebase nicotine is considered bioactive. Freebase nicotine can be absorbed into the bloodstream easily.

When protonated nicotine enters the lungs, it is deposited onto the mucosal layer that covers the alveoli. The pH of the mucosal layer is approximately 7.4. Protonated nicotine slowly deprotonates at this pH. Typically, from 18% to 22% of the protonated nicotine forms freebase nicotine and passes into the bloodstream easily. The remaining from 78% to 82% of the protonated nicotine remains in the monoprotonated form. The monoprotonated form also passes into the bloodstream, but not as effectively as freebase nicotine.

The nicotine generated by the disproportionation process of heated coil systems undergoes a similar process en route to the lungs, and ultimately the bloodstream. Freebase nicotine is more volatile than protonated nicotine, which results in the freebase nicotine becoming “off-gassed” from the aerosolised droplets and being deposited in the mouth and larynx of the upper airway. Owing to the freebase nicotine being deposited in the mouth and larynx of the upper airway, the absorption of the freebase nicotine into the bloodstream is twice as slow as the absorption through the alveoli of the lungs. In contrast, the less volatile protonated nicotine is able to remain in the aerosolised droplets and be inhaled deep into the lungs.

In an example of the invention, ultrasonic devices may be used to achieve aerosolisation. Some non-limiting examples of such ultrasonic devices are provided in PCT application number PCT/IB2019/060810. Typical ultrasonic devices comprise a liquid reservoir structure that comprise a liquid chamber that received the liquid to be atomised, a sonication chamber and a capillary element positioned between the liquid chamber and the sonication chamber. Ultrasonic devices do not heat e-liquids to achieve aerosolisation. Instead, ultrasonic devices use both acoustic cavitation and capillary waves to atomise the e-liquid.

Acoustic cavitation is the growth and implosion of tiny bubbles in a liquid. The size of bubbles formed is dependent on many factors including frequency and the liquid itself, and therefore the size of bubbles formed varies. Typically, the size of the bubbles is on the scale of nanometres to micrometres. The phenomenon of acoustic cavitation is created by high frequency (from 20 kHz to several MHz) sound waves. The sound waves create waves of extremely high and low pressures (several hundred atmospheres) within the liquid, which allows the bubbles to grow and collapse very rapidly. The bubbles typically collapse within microseconds. When the bubbles reach a critical size after a few acoustic cycles, the bubbles rapidly implode. The critical size and number of cycles typically depends on characteristics of the system, such as liquid used. The implosion results in the rapid release of heat as well as a shockwave.

Acoustic cavitation occurs on a nano to micro scale, and therefore all the physical properties occur on the same scale. Acoustic cavitation occurs in nanoseconds or microseconds, over distances of nanometres or micrometres.

The release of the heat is effectively an adiabatic process. The heat dissipates at a speed on the order of 109 K/s (plus or minus one order of magnitude) into the cooler insulating surround liquid.

The shockwave is important in the process of ultrasonic aerosolisation. The shockwave aids the formation of capillary waves at the surface of the liquid. The capillary waves propagate extremely quickly. The speed at which the capillary waves propagate is dependent on the system, such as liquid used. Owing to the speed at which the capillary waves propagate, millions of microscopic droplets are formed. The microscopic droplets break the surface tension of the liquid and are ejected into the air, resulting in aerosolisation of the droplets.

The droplets are from typically from 0.25 to 0.5 microns in size. The droplets form an aerosol which can be absorbed by a user through breathing.

In some examples of the invention, heatless aerosolisation (i.e. ultrasonic aerosolisation) permits the nicotine salt in the e-liquid to remain in the e-liquid as the nicotine salt, without disproportionation (as experienced with heated coil systems). The nicotine salt may be inhaled deep into the lungs. In some examples of the invention, the concentration of nicotine salt inhaled into the lungs is high relative to the concentration of nicotine salt inhaled into the lungs from the use of heated coil devices, since less nicotine is deposited in the upper airway.

Upon entering the lungs, the nicotine component of the nicotine salt may be deprotonated by disproportionation and forms one molecule of diprotonated nicotine and one molecule of unprotonated (freebase) from two molecules of monoprotonated nicotine. The freebase nicotine passes into the bloodstream easily. The remaining protonated nicotine also passes into the bloodstream, but not as effectively as freebase nicotine.

Nicotine travels to the brain after entering the bloodstream. Once in the brain, the nicotine binds to nicotinic acetylcholine receptors (nAchRs), which enhances the flow of sodium and potassium ions through the receptors. The flow of sodium and potassium ions through the receptors results in stimulation of the neurons which the ions are associated with. Stimulation of the neurons results in the release of neurotransmitters, such as dopamine, which causes the “buzz” effect that nicotine users are seeking.

By using ultrasonic devices and a nicotine salt in an e-liquid, the nicotine salt can be inhaled deep into the lungs, and the freebase nicotine can be formed in the lungs where it can easily enter the bloodstream. Through using ultrasonic devices and an e-liquid comprising a nicotine salt, in combination, the user feels an enhanced nicotine effect (compared to the same concentration of a nicotine salt in an e-liquid vaporised by heated coil systems). In other words, by using ultrasonic devices and a nicotine salt (in an e-liquid) in combination, there is a synergistic effect.

Without wishing to be bound by theory, the synergistic effect occurs at least because the use of a nicotine salt in an e-liquid in combination with an ultrasonic device allows the level of nicotine delivered to the lungs to be raised with a relatively lower level of nicotine salt in the e-liquid, without the user feeling a sensory irritation (as they would with a heated coil system). The nicotine salts then enter the lungs, without being deposited in the mouth and larynx of the upper airway owing to the use of ultrasonic devices, where the nicotine salt forms freebase nicotine. Freebase nicotine enters the bloodstream of the user quickly and easily. Owing to increased levels of nicotine entering the bloodstream of the user quickly, the user feels an enhanced nicotine effect with the “throat hit” minimised and/or mitigated.

### EXAMPLES

The following are non-limiting examples that discuss the advantages of using ultrasonic aerosolisation with an e-liquid comprising a nicotine salt.

### Example 1: The Use of Nicotine Levulinate as the Nicotine Salt

In non-limiting examples, four example compositions of e-liquids comprise nicotine, propylene glycol, vegetable glycerin, water and flavourings. The % concentration of each component in the e-liquids is shown in Table 1, Table 2, Table 3 and Table 4.

TABLE 1
The % concentration of each component in the e-liquid (e-liquid 1).

| Component | % (w/w) |
|---|---|
| Propylene glycol | 15.1 |
| Vegetable glycerin | 70 |
| Water | 10 |
| Nicotine | 1.7 |
| Levulinic acid | 0.2 |
| Flavourings | 3 |

TABLE 2
The % concentration of each component in the e-liquid (e-liquid 2).
(Approximately, 2:1 molar ratio of levulinic acid to nicotine.)

| Component | % (w/w) |
|---|---|
| Propylene glycol | 12.87 |
| Vegetable glycerin | 70 |
| Water | 10 |
| Nicotine | 1.7 |
| Levulinic acid | 2.43 |
| Flavourings | 3 |

TABLE 3
The % concentration of each component in the e-liquid (e-liquid 3).
(Approximately, 1:1 molar ratio of levulinic acid to nicotine.)

| Component | % (w/w) |
|---|---|
| Propylene glycol | 14.08 |
| Vegetable glycerin | 70 |
| Water | 10 |
| Nicotine | 1.7 |
| Levulinic acid | 1.22 |
| Flavourings | 3 |

TABLE 4
The % concentration of each component in the e-liquid (e-liquid 4).
(Approximately, 3:1 molar ratio of levulinic acid to nicotine.)

| Component | % (w/w) |
|---|---|
| Propylene glycol | 11.64 |
| Vegetable glycerin | 70 |
| Water | 10 |
| Nicotine | 1.7 |
| Levulinic acid | 3.66 |
| Flavourings | 3 |

In the non-limiting examples, the nicotine in solution is all or part in the form of nicotine levulinate.

The nicotine levulinate salt is formed by combining nicotine and levulinic acid in solution. This results in the formation of the salt nicotine levulinate, which comprises a levulinate anion and a nicotine cation.

The % concentration of nicotine in the e-liquid shown in Table 1, Table 2, Table 3 and Table 4 is approximately equivalent to 17 mg/ml.

The e-liquid is placed into an ultrasonic device. In this non-limiting example, the ultrasonic device is that described in PCT/IB2019/060810. The e-liquid is then aerosolised, and inhaled by the user into the lungs. Users experienced a desired nicotine “buzz” effect with minimal or no “throat hit”.

For nicotine to enter the bloodstream, the nicotine component of the nicotine salt is deprotonated. As discussed in the Chemistry of Nicotine/Levulinic Acid (BN: 511034204-511034215), nicotine levulinate protonates only the pyrrolidine nitrogen of the nicotine molecule. The protonation results in the formation of a monoprotonated nicotine molecule. A proportion of the monoprotonated nicotine is deprotonated and enters the bloodstream as freebase nicotine; another proportion of the monoprotonated nicotine enters the bloodstream as monoprotonated nicotine. The monoprotonated nicotine does not enter the bloodstream as effectively as the freebase nicotine (Lippiello et al.).

With reference to the different compositions of Tables 2, 3 and 4, all three examples provide a beneficial reduction in “throat hit”. Therefore, compositions comprising any one of a 1:1, a 2:1 or a 3:1 molar ratio of levulinic acid (or other organic acid) to nicotine provide beneficial effects.

Nicotine molecules contain two nitrogen atoms, one in the pyridine ring and the other in the pyrrolidine ring. These two nitrogen atoms both have free electron pairs in the freebase form. These two nitrogen atoms can accept donor molecules, such as the protons from the hydroxyls of levulinic acid (or other organic acids). The nitrogen atom of the pyrrolidine ring nitrogen will be the first nitrogen to accept a proton from the levulinic acid (or other organic acid), followed by the pyridine ring nitrogen. To protonate both nitrogen atoms on a nicotine molecule, two molar equivalents of levulinic acid (or other organic acid) are necessary.

The different ratios of levulinic acid to nicotine shown in Tables 2, 3 and 4 were tested to see if different levels of equivalent acid produced different effects. Users reported that composition with a 1:1 molar ratio (i.e. the composition of Table 3) still delivers a reduced throat hit. However, the 2:1 molar ratio (i.e. the composition of Table 2) and the 3:1 molar ratio (i.e. the composition of Table 4) offers a further throat hit reduction. This result indicates that using a 1:1 molar ratio (or higher amount) of levulinic acid (or other organic acid) to nicotine provides beneficial effects (i.e. with the levulinic acid being the greater component for ratios higher than 1:1).

Advantageously, the nicotine levulinate comprises the levulinate anion. The levulinate anion (as a component of the nicotine levulinate) has an octanol:water partitioning coefficient (P) of 0.69, this is a 500-fold increase in comparison to levulinic acid (P=0.00145), as exemplified in Table 5.

TABLE 5
Octanol:Water Partitioning Coefficient Data for
Nicotine, Nicotine Levulinate and Levulinic Acid.

| Solute | logP¹ | P | % Nonpolar | % Aqueous² |
|---|---|---|---|---|
| Nicotine | 0.45 | 2.82 | 74 | 26 |
| Nicotine Levulinate | −0.16 | 0.69 | 41 | 59 |
| Levulinic Acid | −2.84 | 0.00145 | 0.15 | 99.85 |

¹P = [solute in octanol]:[solute in aqueous buffer]
²Phosphate buffer, pH 7.4

The partitioning coefficient can be used as a measure of lipid solubility. Owing to the levulinate anion having a high lipid solubility, the levulinate anion will pass into the bloodstream with the nicotine.

Once the nicotine and levulinate anion have entered the bloodstream, the nicotine and levulinate anion travel to the brain.

The additional presence of the levulinate ion increases the amount of nicotine that binds to receptors in the brain (Lippiello et al.). The levulinate anion increases the amount of nicotine binding to receptors in the brain in two ways. One way is through increasing the affinity of receptor sites to the nicotine molecule, or secondly, by causing positive binding cooperativity of nicotine at an additional class of receptor sites.

The presence of levulinate anions therefore results in more nicotine binding to receptors in the brain. As discussed in Lippello et al., the proportion of nicotine binding sites can increase by 20-50% when nicotine levulinate is inhaled, compared to other nicotine salts such as nicotine salicylate.

The use of ultrasonic devices to aerosolise a nicotine salt e-liquid comprising nicotine levulinate leads to an enhanced nicotine effect on a user, with a relatively low (compared to heated coil devices) concentration of nicotine in the e-liquid.

The use of ultrasonic devices to aerosolise a nicotine salt e-liquid with the use of nicotine levulinate results in the device delivering a nicotine experience that is unparalleled by any of the current heated coil ENDS on the market.

A similar effect is found when nicotine levulinate is replaced in whole or in part by another nicotine salt, including but not limited to nicotine benzoate, nicotine maleate, nicotine ditartrate, nicotine salicylate, nicotine citrate and nicotine lactate.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

### BIBLIOGRAPHY

The following documents are incorporated herein by reference in their entirety:
Lippiello, P. M., Femandes, K. G., Reynolds, J. H., & Hayes, A. W. (1989, September 25). Enhancement of nicotine binding to nicotinic receptors by nicotine levulinate and levulinic acid. R. J. Reynolds. Bates No. 509336913-509336640. Retrieved from http://tobacco-documents.org/product_design/509336913-6940.html.
Talih S, Salman R, El-Hage R, et al. Characteristics and toxicant emissions of JUUL electronic cigarettes. Tob Control. 2019. doi: 10.1136/tobaccocontrol-2018-054616
Seeman J., Fournier J., Paine III J., Waymack B. The Form of Nicotine in Tobacco. Thermal Transfer of Nicotine and Nicotine Acid Salts to Nicotine in the Gas Phase. Journal of Agricultural Food Chemistry. 1999.
RJ Reynolds Records. Chemistry of Nicotine/Levulinic Acid. 1992. BN: 511034204-511034215. Retrieved from https://www.industrydocuments.ucsf.edu/docs/hfdy0046

What is claimed is:

1. An e-liquid composition for use with an ultrasonic device, comprising:
a nicotine salt.

2. The e-liquid composition of claim 1, wherein the e-liquid composition further comprises one, two, three or four of:
propylene glycol;
vegetable glycerin;
water; and,
flavourings.

3. The e-liquid composition of claim 2, wherein:
A. the relative amount of vegetable glycerin in the composition is: from 55 to 80% (w/w), or from 60 to 80% (w/w), or from 65 to 75% (w/w), or 70% (w/w); and/or,
B. the relative amount of propylene glycol in the composition is: from 5 to 30% (w/w), or from 10 to 30% (w/w), or from 15 to 25% (w/w), or 20% (w/w); and/or,
C. the relative amount of water in the composition is: from 5 to 15% (w/w), or from 7 to 12% (w/w), or 10% (w/w); and/or,
D. the amount of nicotine and/or nicotine salt in the composition is: from 0.1 to 80 mg/ml, or from 0.1 to 50 mg/ml, or from 1 to 25 mg/ml, or from 10 to 20 mg/ml, or 17 mg/ml.

4. The e-liquid composition of claim 2, wherein the composition comprises, or consists of, (in % (w/w)):

| | |
|---|---|
| propylene glycol | from 10 to 20 |
| vegetable glycerin | from 65 to 75 |
| water | from 5 to 15 |
| nicotine | from 1 to 5 |
| organic acid | from 0.1 to 5.0 |
| flavourings | balance; |

wherein the organic acid comprises, or consists of: benzoic acid, levulinic acid, malic acid, tartaric acid, salicylic acid, citric acid or lactic acid, or any combination of any two, three, four, five, six of seven of these organic acids.

5. The e-liquid composition of claim 2, wherein the composition comprises (in % (w/w)):
organic acid from 0.1 to 5.0; or, from 1.0 to 4.0; or, from 0.1 to 2.0.

6. The e-liquid composition of claim 2, wherein the composition comprises, or consists of, (in % (w/w)):

| | |
|---|---|
| propylene glycol | from 11 to 16 |
| vegetable glycerin | from 69 to 71 |
| water | from 9 to 11 |
| nicotine | from 1 to 3 |
| levulinic acid | from 0.1 to 4.0 |
| flavourings | balance. |

7. The e-liquid composition of claim 6, wherein the composition comprises (in % (w/w)):
propylene glycol from 14 to 16.

8. The e-liquid composition of claim 1, wherein the composition comprises (in % (w/w)):
levulinic acid from 1.0 to 4.0; or, from 0.1 to 1.0; or, from 0.1 to 0.5.

9. The e-liquid composition of claim 6, wherein the composition comprises (in % (w/w)):

| | |
|---|---|
| propylene glycol | 15.1 |
| vegetable glycerin | 70 |
| water | 10 |
| nicotine | 1.7 |
| levulinic acid | 0.2 |
| flavourings | 3; or, |
| propylene glycol | 12.87 |
| vegetable glycerin | 70 |
| water | 10 |
| nicotine | 1.7 |
| levulinic acid | 2.43 |
| flavourings | 3; or, |
| propylene glycol | 14.08 |
| vegetable glycerin | 70 |
| water | 10 |
| nicotine | 1.7 |
| levulinic acid | 1.22 |
| flavourings | 3; or, |
| propylene glycol | 11.64 |
| vegetable glycerin | 70 |
| water | 10 |
| nicotine | 1.7 |
| levulinic acid | 3.66 |
| flavourings | 3. |

10. The e-liquid composition of claim 1, wherein the nicotine salt is selected from the group consisting of:
nicotine benzoate, nicotine lactate, nicotine maleate, nicotine ditartrate, nicotine salicylate, nicotine citrate and nicotine levulinate, or any combination of any two, three, four, five, six or seven of these nicotine salts.

11. The e-liquid composition of claim 1, wherein the nicotine salt is nicotine levulinate.

12. The e-liquid composition of claim 10, wherein the molar ratio of nicotine to organic acid salt (nicotine:organic acid salt) is: 1:1 or greater; or, 1:2 or greater; or, from 1:1 to 1:4; or, from 1:1 to 1:3.

13. The use of an e-liquid composition in providing nicotine to a user, the use comprising:
providing an e-liquid composition comprising a nicotine salt and at least one of propylene glycol; vegetable glycerin; water; and, flavourings;
placing the e-liquid composition in an ultrasonic device; and,
aerosolising the e-liquid composition in the ultrasonic device.

14. The use of claim 13, wherein the use further comprises:
inhaling the aerosolised composition.

15. The use of claim 13, wherein the ultrasonic device is an ultrasonic mist inhaler, comprising:
a liquid reservoir structure comprising a liquid chamber adapted to receive liquid to be atomized,
a sonication chamber in fluid communication with the liquid chamber,
a capillary element arranged between the liquid chamber and the sonication chamber.

16. The use of claim 15, wherein the capillary element is a material at least partly in bamboo fibers; optionally,
wherein the capillary element material is 100% bamboo fiber; or, wherein the capillary element material is at least 75% bamboo fiber and, preferably, 25% cotton; and/or,
wherein the capillary element is of a thickness between 0.27 mm and 0.32 mm and, preferably, has a density between 38 g/m² and 48 g/m².

17. The use of claim 15, wherein the capillary element has a flat shape.

18. The use of claim 15, wherein the capillary element comprises a central portion and a peripheral portion; optionally,
wherein the peripheral portion has an L-shape cross section extending down to the liquid chamber; and/or
wherein the central portion has a U-shape cross section extending down to the sonication chamber.

19. A method of delivering a nicotine salt to a user, the method comprising:
providing an e-liquid composition comprising a nicotine salt and at least one of propylene glycol; vegetable glycerin; water; and, flavourings;
placing the e-liquid composition in an ultrasonic device; and,
aerosolising the e-liquid composition in the ultrasonic device.

20. The method of claim 19, wherein the method further comprises:
inhaling the aerosolised composition.
